(19) United States
(12) Patent Application Publication
(10) Pub. No.: US 2013/0072913 A1
(43) Pub. Date: Mar. 21, 2013

(54) MEDICAL DEVICE FOR SURGERY

(71) Applicants: Byung Ju Yi, Gyeonggi-do (KR); Young Sik Kwon, Gyeonggi-do (KR)

(72) Inventors: Byung Ju Yi, Gyeonggi-do (KR); Young Sik Kwon, Gyeonggi-do (KR)

(73) Assignee: INDUSTRY-UNIVERSITY COOPERATION FOUNDATION HANYANG UNIVERSITY ERICA CAMPUS, Gyeonggi-do (KR)

(21) Appl. No.: 13/698,355

(22) PCT Filed: Mar. 25, 2011
(86) PCT No.: PCT/KR11/02066

§ 371 Date: Nov. 16, 2012

(30) Foreign Application Priority Data
May 17, 2010 (KR) 10-2010-0045830
Mar. 11, 2011 (KR) 10-2011-0021780

(51) Int. Cl. A61B 17/00

(52) U.S. Cl. CPC A61B 17/00
USPC 606/1

(57) ABSTRACT

Disclosed herein is a medical device for surgery according to the present invention including: a frame which is led into a body and the frame's front end portion is fixed adjacent to an affected part, and having a route of travel inside; at least one treatment unit moving along the route of travel of the frame to be accessible to the affected part and treating the affected part; a driving unit generating a driving force to drive the at least one treatment unit; and a control unit controlling the driving unit generated from the control unit to control operation of the treatment unit. According to the present invention, accessibility to the affected part is improved as compared to that of the case according to the related art, and the treatment unit performs treatment on the affected part in the state in which the frame is fixed in the region adjacent to the affected part, thereby making it possible to improve accuracy of surgery.

### TECHNICAL FIELD

This present invention relates to a medical device for surgery, and more specifically, to a medical device for surgery capable of improving accessibility to an affected part of a patient and performing a surgery on the affected part using a treatment unit, or capable of being easily deformed or bended according to a shape of an inner portion of a body and maintained in the deformed or bended state, making it possible to reduce the patient's burden and enhance accuracy of the surgery.

### BACKGROUND ART

At the time when a wound, a cystis, or the like, has been generated in a larynx, a large intestine, or the like in a body, a surgical device for cutting out is led into the body to perform a surgery for treating the wound or removing the cystis, or so forth.

Particularly, if the wound has been generated in the larynx, it should be removed through a suspension laryngeal surgery. Here, in order for a suspension laryngoscope, which is a kind of medical device for surgery, to approach the affected part, its route of travel to the affected part may be secured in a straight-line shape.

FIG. 1 is a view showing the state in which a medical device for surgery according to the related art is led into a larynx in a body. As shown in FIG. 1, a route of travel formed in a straight-line shape is secured by leaning backward the head of a patient, who is a subject to the surgery, and then the medical device for surgery 1 is led in to remove the affected part in the larynx 3.

However, in the case in which the head of the patient is leaned backward for surgery as described above, incidental body damage may occur due to pressure of a neck of the patient or the surgery may not be not able to be performed according to physical characteristics of the patient.

About 10% of the patients who need the above-mentioned surgery may not expose the affected part in the larynx 3 even when they lean their heads backward as shown in FIG. 1. Therefore, there is a limitation that the affected part in the larynx 3 may not removed by the surgery.

Particularly, in the case of the patient being a male patient, or having a thick or short neck, an overweight, a small under jaw, tongue, or the like, a neck disk, or a swinging tooth or a false tooth, it is not easy to perform the surgery described above. In addition, even though the surgery is performed, other complications, such as a tooth damage, a laryngeal edema, or the like, may be occur.

In addition, in the related art a surgeon performs the surgery on the affected part while seeing the state of the affected state with his or her eyes as shown in FIG. 1. In this case, if the surgeon has a tremor in his or her hands or does not find the accurate position of the affected part, the surgery may not be accurately performed.

Therefore, development of a medical device for surgery having a new structure capable of removing an affected part in a larynx, or the like, in a non-backward-leaning position, that is, an comfortable position, and capable of reliably performing the surgery on the affected part been urgently demanded.

In addition, the demand for a medical device for surgery capable of easily accessing to a position of an affected part in a patient without burdening the patient's neck or body and capable of improving accuracy of the surgery by maintaining a certain state during the surgery has been increased.

### DISCLOSURE

### Technical Problem

According to the present invention, a surgery is performed on an affected part in a patient using a treatment unit in a state in which a frame is fixed. Therefore the present invention may provide a medical device for surgery capable of preventing accuracy of the surgery from deteriorating due to incidental factors such as tremor of a surgeon, or the like.

The treatment unit which substantially performs the surgery on the affected part may operate flexibly according to the position of the affected part. Therefore, the present invention may provide a medical device for surgery capable of improving ease of the surgery on the affected part.

According to the present invention, accessibility to the affected part may be improved and the surgery may be performed on the affected part using the treatment unit while a state in which it is inserted into the body is being stably maintained. Therefore the present invention provides a medical device for surgery capable of improving accuracy of the surgery.

According to the present invention, a frame is maintained in a state in which its shape is fixed or deformed or bent corresponding to an inner shape of the body. Therefore, the present invention provides a medical device for surgery capable of preventing incident damage from occurring by relieving pressure applied to a certain part of the body.

The present invention provides a medical device for surgery capable of deforming the shape of the frame according to a shape of an inner part of the body approaching the affected part or adjusting a degree of bending thereof.

The present invention provides a medical device for surgery capable of improving stability of the surgery by maintaining the state in which the frame is deformed or bended during the surgery on the affected part.

### Technical Solution

To solve the problems described above, an object of the present invention is to provide a medical device for surgery, including: a frame which is led into a body and the frame's front end portion is fixed adjacent to an affected part, and having a route of travel inside; at least one treatment unit moving along the route of travel of the frame to be accessible to the affected part and treating the affected part; a driving unit generating a driving force to drive the at least one treatment unit; and a control unit controlling the driving unit generated from the control unit to control operation of the treatment unit.

The frame may have a bent portion bending corresponding to the inner portion of the body in a fixed form.

The frame may be formed so as to be deformed or bent according to a shape of the inner portion of the body.

The frame may include a plurality of variable members provided to be overlapped to each other so that the shape of the frame may be deformed or bent by the shape of inner portion of the body contacting an outer surface of the frame while the frame is being led into the inner portion of the body.

One of the both ends in a length direction of the frame may be inserted into an inside of a neighboring variable member, and an outside of the neighboring variable member may be inserted at the other.

The variable member may further have protrusions protruding toward the route of travel, and the protrusion has a hole in which an operation wire is inserted.

The protrusions may be formed to face each other toward the upper and lower portion of the route of travel, or formed to protrude in the direction perpendicular to a line passing through the center of the route of travel.

One end of the operation wire may pass through the holes formed in the protrusions to be connected to a frame cap mounted in the front end of the frame or one end of the frame, and the other may be provided to the other end of the frame to be connected to bending adjusting portion adjusting a degree of bending of the variable member.

The bending adjusting portion may have a locking means for maintaining a deformed state or a bent state of the variable member.

The locking means may prevent the frame from unbending by seizing the operation wire in the state in which the frame is bent by the bending adjusting portion pulling one of the operation wires.

The locking means may contact the variable member adjacent to the bending adjusting portion in the state of seizing the operation wire.

The locking means may include a clamp seizing the operation wire or a hook caught by a groove formed in the operation wire.

The locking wire may be able to seize or release the operation wire and maintains the state of seizing the operation wire while the treatment unit is treating the affected part.

Here, the treatment unit may include: a treatment portion performing a surgery on the affected part; and a moving guide portion coupled to the treatment portion by its front and capable of not only having a length direction so as to move along the route of travel but also flexibly operating.

The moving guide portion may be provided as a spring capable of a position change in a length direction and elastic deformation in a lateral direction or made of a flexible plastic or steel material capable of elastic deformation.

The moving guide portion may have a plurality of link members coupling connected in the length direction, and the plurality of link members rotates in multi directions based on a joint portion.

The treatment portion may include: a connecting member rotatably coupled to a front end of the moving guide portion in a lateral direction of the moving guide portion and a traverse direction with respect to the moving guide portion; a rotating member rotatably coupled to the connecting member; and a treating member coupled to the rotating member and directly performing treatment on the affected part, and the connecting member, the rotating member, and the treating member may operate by a driving force generated by the driving unit based on a command signal of the control unit.

The treating member may include one of a grip for seizing the affected part, scissors for removing the affected part, a knife for cutting out the affected part, a snare for enclosing and cutting out the affected part, a cautery for removing the affected part, and a sucking tube for sucking the removed affected part.

The driving unit may include: a front-back moving member moving a mounting member in which a rear end portion of the treatment unit is mounted forward and backward on the route of travel of the frame; and a wire-type treatment portion driving member connected to the connecting member, the rotating member, and the treating member, portion and controlling operation of a selected member among the connecting member, the rotating member, and the treating member based on the command signal of the control unit.

The medical device for surgery may further comprises a photographing unit which moves along the route of travel of the frame to approach the affected part and photograph a state of the affected part, and a control unit controls operation of the treatment unit based on photographing information of the photographing unit.

### Advantageous Effects

As set forth above, in the medical device for surgery according to exemplary embodiments of the present invention, accessibility to the affected part may be improved as compared to the case of the related art and the treatment unit may treat the affected part in the state in which the frame is fixed in the region adjacent to the affected part or in the state in which the shape of the frame or the degree of bending of the frame is maintained in the region adjacent to the affected part, making it possible to improve accuracy of the surgery.

In addition, in the medical device for surgery according to exemplary embodiments of the present invention, the frame may be provided in the shape corresponding to the inner shape of the body or be deformed corresponding to the inner shape of the body, such that the patient does not have to adopt a specific pose for surgery, for example, a pose to lean his or her head backward. Therefore, Applying pressure on a certain part of the body is prevented, thereby making it possible to prevent incidental body damage from occurring.

In addition, in the medical device for surgery according to exemplary embodiments of the present invention, the surgery may be performed on the affected part using the treatment unit in the state in which the frame is fixed or in the state in which the frame is maintained in the deformed or bended shape, making it possible to prevent accuracy of the surgery from deteriorate due to incidental factors, such as tremor of hands of the operator.

In addition, in the medical device for surgery according to exemplary embodiments of the present invention, the treatment unit which substantially performs the surgery on the affected part may operate flexibly according to the position of the affected part, making it possible to improve ease of the surgery on the affected part.

### DESCRIPTION OF DRAWINGS

FIG. 1 is a view showing a state in which a medical device for surgery according to the related art is led into a larynx in a body.

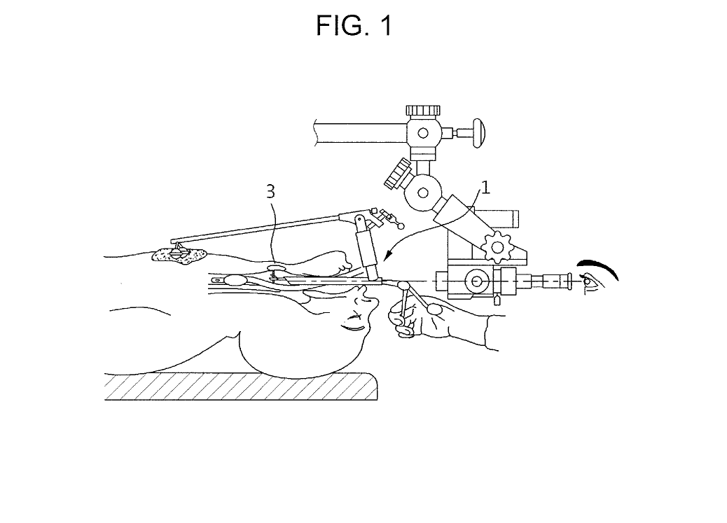

FIG. 2 is a view showing a state in which a medical device for surgery according to an exemplary embodiment of the present invention is led into a larynx in a body.

FIG. 3 is a perspective view of the medical device for surgery shown in FIG. 2.

FIG. 4 is a perspective view of a frame shown in FIG. 3.

FIG. 5 is a view for explaining a multi-degree-of-freedom surgery of a treatment unit shown in FIG. 3.

FIG. 6 is a perspective view showing a frame of a medical device for surgery according to another exemplary embodiment of the present invention.

FIG. 7 is a cross-sectional view showing a variable member of the frame shown in FIG. 6.

FIG. 8 is a partially cross-sectional view showing the frame in a state in which it is bended by the variable member shown in FIG. 7.

### MODE FOR INVENTION

Hereinafter, exemplary embodiments according to the present invention will be described in detail with reference to the accompanying drawings. However, the present invention will be not limited or restricted to the exemplary embodiments. Like reference numerals proposed in each drawing denote like components.

The explanation below is one of many aspects of the present invention, and the description below forms part of the detailed description of the present invention.

However, hereinafter, although the present invention will be described with respect to the medical device for surgery for removing an affected part generated in a larynx, the present invention is not limited thereto, and it should be understood that the medical device of the present invention may be used to remove affected parts generated in other parts of the body than the affected part in the larynx.

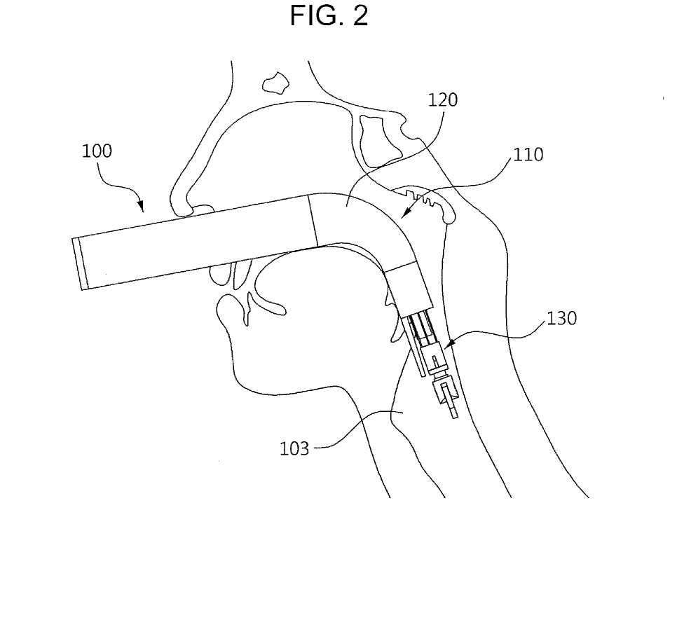

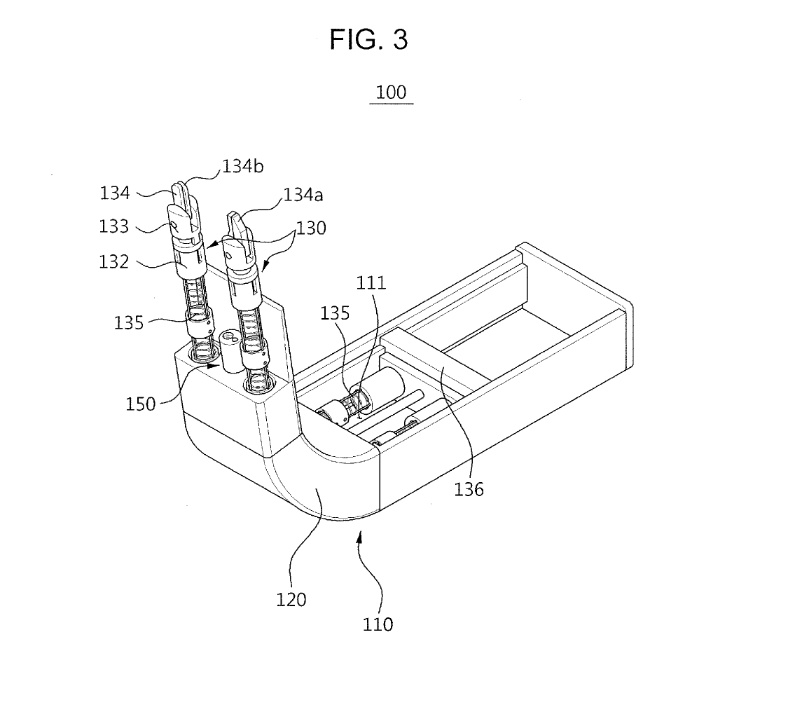

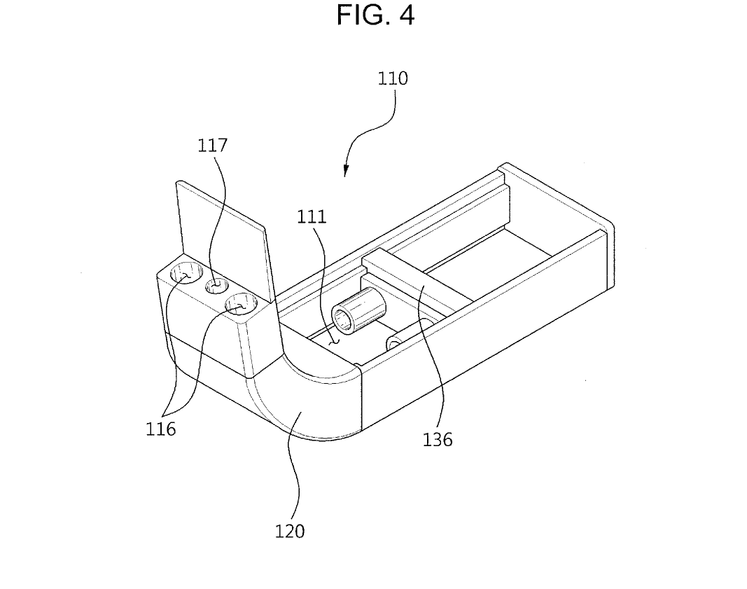

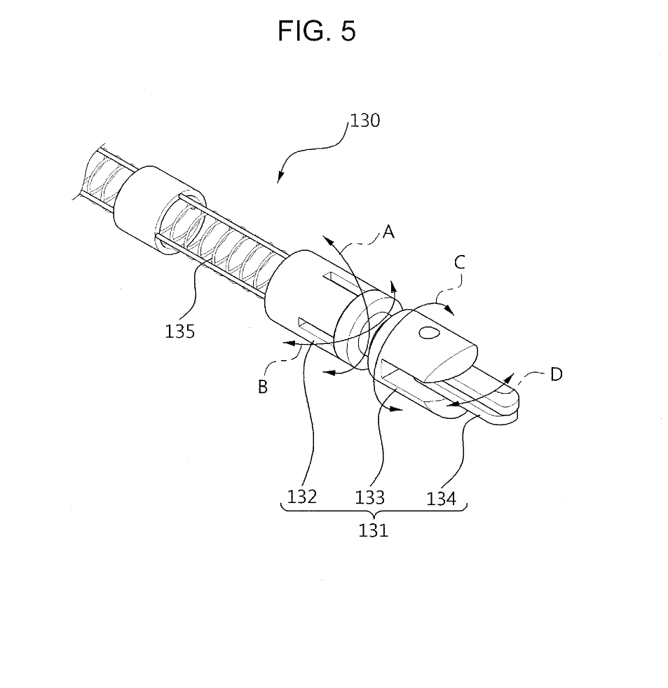

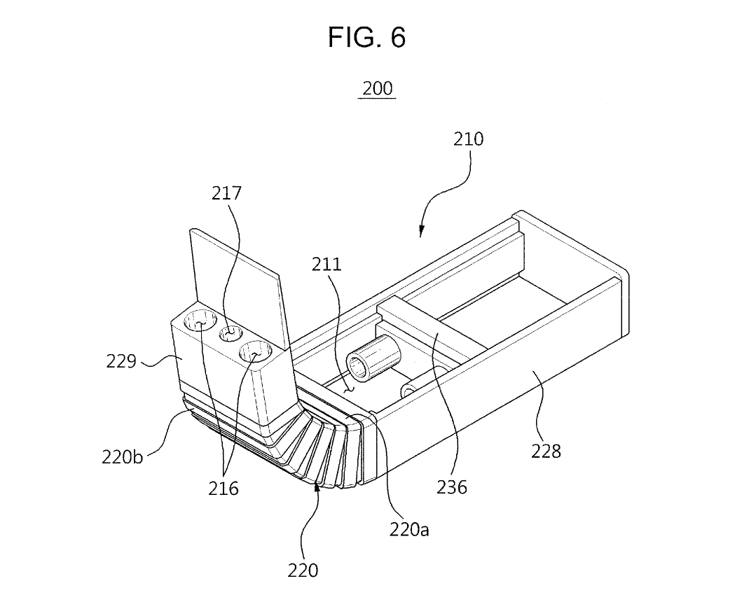

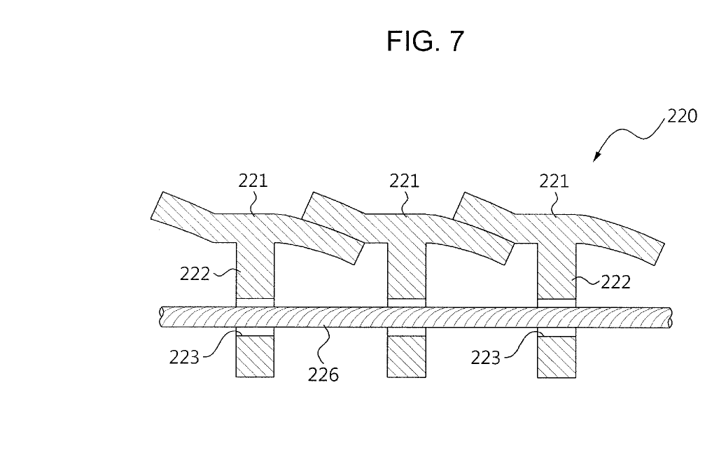

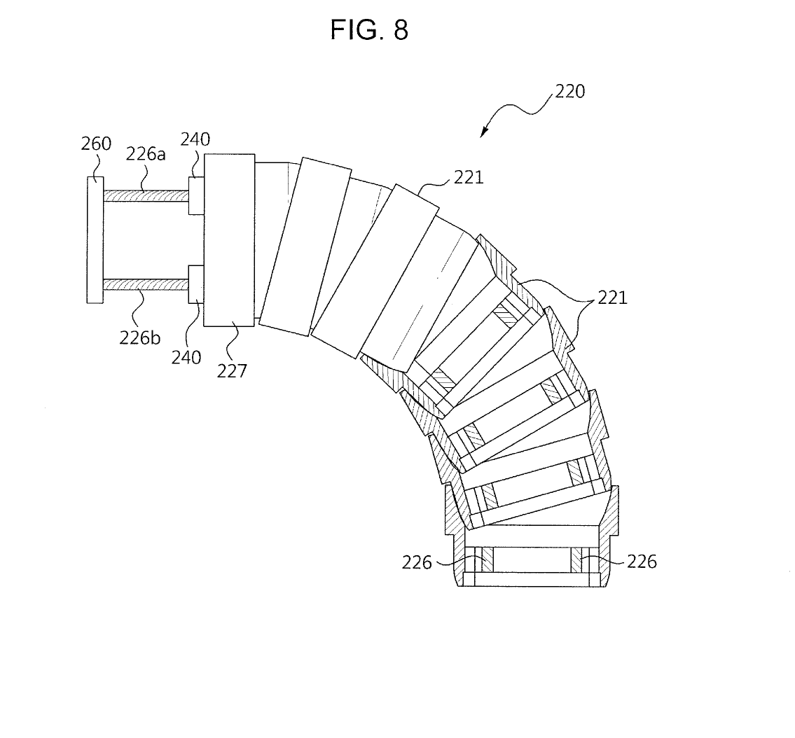

FIG. 2 is a view showing a state in which a medical device for surgery according to an exemplary embodiment of the present invention is led into a larynx in a body, FIG. 3 is a perspective view of the medical device for surgery shown in FIG. 2, FIG. 4 is a perspective view of a frame shown in FIG. 3, FIG. 5 is a view for explaining a multi-degree-of-freedom surgery of a treatment unit shown in FIG. 3, FIG. 6 is a perspective view showing a frame of a medical device for surgery according to another exemplary embodiment of the present invention, FIG. 7 is a cross-sectional view showing a variable member of the frame shown in FIG. 6, and FIG. 8 is a partially cross-sectional view showing the frame in a state in which it is bended by the variable member shown in FIG. 7.

As shown in these figures, the medical device for surgery 100 according to an exemplary embodiment of the present invention is configured to include a frame 110 which is led into the larynx 103 through a mouth to fix its front end portion adjacent to the affected part generated in the larynx 103 and inside which a route of travel 111 is formed, a plurality of treatment unit 130 which moves along the route of travel 111 of the frame 110 to approach the affected part and directly treats the affected part, a photographing unit 150 which moves along the route of travel of the frame 110 to approach the affected part and photograph a state of the affected part, a control unit (now shown) which controls operations of the treatment units 130 based on photographing information of the photographing unit 150.

More specifically, first, the frame 110 is formed with a bent portion 120 bent corresponding to shapes of the mouth and larynx 103 as shown in FIGS. 2 to 4. Therefore, a patient does not have to adopt a specific pose, for example, a pose that the patient leans his or her head backward as in the case according to the related art, whereby the patient may maintain a comfortable pose during the surgery.

The frame 110 is fixed in the body, allowing the treatment unit 130 to move into the body. Referring to FIG. 4, at the front end portion of the frame 110 a through hole 116 movably supporting the end portion of the treatment unit 130 and a through hole 117 movably supporting photographing unit 150 are formed to be long in a length direction.

That is, the treat unit 130 may be led into the inner portion of the frame 110 through the rear end portion of the frame 110, and then move along the through hole 116 provided at the front end portion to approach the affected part generated in the larynx 103.

However, since in the present exemplary embodiment the case in which the affected part in the larynx 103 is treated has been explained, the frame 110 has been described as having a bent shape, but the present invention is not limited thereto and it should be understood that the frame 110 may have other shapes according to parts in which affected parts are generated, for example, a linear shape.

Meanwhile, the treatment unit 130, which is the unit that directly operates on the affected part, includes a treatment portion 131 directly dealing with the affected part, and a moving guide portion 135 having a front end coupled to the treatment portion 131 and provided in a long shape to move along the through hole 116 of the frame 110, and a driving unit (now shown) driving the treatment portion 131 and moving the moving guide portion 135 based on a command signal of a control unit, as shown in FIGS. 2 and 3.

Describing the moving guide portion 135 prior to the treatment portion 131, the moving guide portion 135 of the present exemplary embodiment may be elastically deformed in a length direction and a lateral direction, such that a spring having a flexible property may be used as the moving guide portion 135.

Therefore, the moving guide portion 135 may not only move smoothly forward and backward along the through hole 116 by the driving unit which is described below, and may be also flexibly bent at the bent portion 120 of the frame 110, thereby allowing the treatment portion 131 mounted at the front end of the moving guide portion 135 to approach the affected part smoothly.

However, although in the present exemplary embodiment the moving guide portion 135 is described to be provided in a spring type, the present invention is not limited, and it should be understood that the moving guide portion 135 may have a joint-type link structure in which a plurality of link members are coupled to one another in the length direction In addition, the moving guide portion may be made of a flexible plastic or steel material having a predetermined elasticity.

Meanwhile, the treatment portion 131 of the present exemplary embodiment includes a connecting member 132 rotatably coupled to the front end portion of the moving guide portion 135 in a lateral direction (A direction in FIG. 5) or a traverse direction (B direction in FIG. 5) with respect to the moving guide portion 135, a rotating member relatively rotatably coupled to a front end portion of the connecting portion 132 in a C direction in FIG. 5, and a treating member 134 coupled to a front end portion of the rotating member 133 to perform operations for treatment on the affected part (folding operations in a D direction of FIG. 5), as shown in FIG. 5.

The connecting member 132 is rotated in the lateral direction or traverse direction with respect to the moving guide portion 135. In other words, the connecting member 132 may be leaned toward one side with respect to the moving guide portion 135.

Therefore, the treating member 134 may approach the affected part according to the operations of the connecting member 132 in the lateral direction regardless of the position in which the affected part is generated in the larynx 103, thereby making it possible to smoothly perform the surgery on the affected part.

The rotating member 133 is part that connects the connecting member 132 to the treating member 134. The rotating member 133 described above may be relatively rotated with respect to the connecting member 132, allowing the treating member 134 having approached the affected part by the connecting member 132 to be rotated to an appropriate angle for the surgery on the affected part. That is, the treating member 134 positioned at the outmost front end of the treatment portion 131 may approach the affected part by the operation of the connecting member 132 and then be position-converted in an appropriate state for the surgery on the affected part by the operations of the rotating member 133.

The treating member 134, which is a component for directly operating the affected part as shown in FIGS. 2 and 3, may have a grip 134a for seizing the affected part, scissors for removing the affected part, or the like. Therefore, the treating member 134 has to be able to grip and scissor the affected part, which may be driven by a driving force supplied from the driving unit described below.

However, although in the present exemplary embodiment the grip or the scissors have been described, the present invention is not limited thereto and it should be understood that a knife for cutting out the affected part, a snare for enclosing and cutting out the affected part, a cautery for removing the affected part, a sucking tube for sucking the removed affected part, or the like may be provided.

Meanwhile, the photographing unit 150 may move along the route of travel of frame 110 and approach the affected part through the through hole 117 provided at the front end portion of the frame 110, as shown in FIG. 3. The above-mentioned photographing unit 150 may include a lighting member (not shown) for lightening a region of the affected part and a photographing member (not shown) for photographing the region of the affected part in real time.

Due to the above-mentioned configuration, information on the photographed affected part may be monitored through an external monitor. Therefore, the operator may reliably perform the surgery on the affected part while monitoring the state of the affected part.

Here, a body of the photographing unit 150 may be made of a flexible material so that the photographing unit 150 may smoothly approach the region of the affected part. Therefore, the photographing unit 150 may move along the route of travel of the frame 110 as described above, which allows the photographing unit 150 to smoothly approach the region of the affected part.

The control unit serves to control driving of the driving unit described below based on the information obtained by the photographing unit 150. In other words; the photographing unit 150 photographs the region of the affected part in real time and the photographed information is displayed through the external monitor. Here, the operator transfers a command signal to the driving unit and controls the operations of the treatment unit 130. For example, the control unit may drive the driving unit to not only advance but also rotate the treatment unit 130 as a whole. In addition, the control unit may control the treating member 134 provided at the front end of the treatment unit 130 to perform the surgery on the affected part.

Meanwhile, the driving unit includes a front-back moving member (not shown) moving a mounting member 136 in which a rear end portion of the moving guide portion 135 is mounted forward and backward on the route of travel of the frame 110, and a wire-type treatment driving member (now shown) connected to the treatment portion 131 and controlling operations of the treatment portion 131 based on the command signal of the control unit.

The front-back moving member generates the driving force capable of moving the mounting member 136 along the route of travel of the frame 110, thereby making it possible to move the treatment unit 130 coupled to the mounting member 136 forward and backward. That is, in the case in which the front-back moving member generates the driving force advancing the mounting member 136 based on the command signal of the control unit, the treatment unit 130 may move forward. On the other hand, in the case in which the front-back moving member 136 generates the driving force backing up the mounting member 136, the treatment unit 130 may move backward in the inner portion of the frame 110.

The treatment portion driving member, which is provided as a wire type, is connected to the treating member 134, the connecting member 132 and the rotating member 133, respectively, all of which the treatment portion 131 includes. In addition, in the case in which the command signal is supplied from the control unit, the wire-type treatment portion driving member selectively operates the treating member 134, the connecting member 132, or the rotating member 133 to allow the operator to perform the surgery on the affected part with his own intention.

Meanwhile, hereinafter, a process of removing the affected part the larynx 103 will be described using the medical device for surgery 100 having the structure as described above.

First, the frame 110 is led into the mouth so that the front end thereof may be positioned in the larynx 103. Here, the bent portion 120 of the frame 110 is placed at a bent spot between the mouth and the throat, whereby the patient does not have to adopt a specific pose, for example, lean his or her head backward.

Next, the photographing unit 150 and a pair of the treatment units 130 are led into the inner portion of the frame 110. Then, the photographing unit 150 and the pair of the treatment units 130 approach the affected part by the driving force generated by the driving unit.

Then, the affected part is photographed using the photographing unit 150 to obtain photographing information. Here, the photographing information is displayed on the external monitor in real time and the operator operates the treatment unit 130 based on the photographing information and perform the surgery on the affected part.

More specifically, the treatment portion driving member of the driving unit is driven based on the command signal from the control unit to selectively operate each of components of the treatment portion, that is, the treating member 134, the connecting member 132, or the rotating member 133, performing the surgery on the affected part. Here, the treating member 134a included in one of the pair of treatment unit 130, that is, the grip 134a seizes the affected part, and the treating member 134b included in the other of the pair of treatment unit 130, that is scissors 134b cuts out the affected part. Through this process, the surgery on the affected part, for example cutting-out of the affected part may be performed.

As described above, according to the present exemplary embodiment, for example, at the time of performing the surgery on the affected part in the larynx 103, the patient does not have to lean his or her head backward, such that the accessibility to the affected part may be improved. In addition, since the surgery is performed in the state in which the frame 110 is fixed, accuracy of the surgery may be also improved.

In addition, according to an exemplary embodiment of the present invention, the frame is provided in the shape corresponding to the inner shape of the body; whereby the patient does not have to adopt a specific pose, for example, lean his or her head backward. Therefore applying pressure on a certain part of the body is prevented, whereby the incidental body damage may be prevented.

In addition, the treatment unit 130 which substantially performs the surgery on the affected part may operate flexibly according to the position of the affected part, whereby ease of the surgery on the affected part may be improved.

Meanwhile, hereinafter, a medical device for surgery according to another exemplary embodiment of the present invention will be described with reference to drawings.

As shown in FIG. 6, the medical device for surgery 200 according to another exemplary embodiment of the present invention may be configured to include a frame 211 which is led into the larynx 103 (see FIG. 2) through a mouth to fix its front end portion adjacent to the affected part generated in the larynx 103 and inside which a route of travel 211 is formed, a plurality of treatment unit 130 (see FIG. 3) which moves along the route of travel 211 formed inside the frame 210 to approach the affected part and directly treats the affected part, a photographing unit 150 (see FIG. 3) which moves along the route of travel 211 formed inside the frame 210 to approach the affected part and photograph a state of the affected part, a control unit (now shown) which controls operations of the treatment units 130 based on photographing information of the photographing unit 150.

Here, since the treatment unit 130 has the same structure as that of the medical device for surgery according to the exemplary embodiment of the present invention described above, a description thereof will be omitted.

Describing each configuration, the frame may include a bent portion 220 bent corresponding to the shapes of a mouth and the larynx 103. Therefore, a patient does not have to adopt a specific pose, for example, a pose that the patient leans his or her head backward as in the case according to the related art, whereby the frame 110 may easily approach the affected part in the state in which the patient maintains a comfortable pose during the surgery.

The above-mentioned frame 210 maintains its state after being led into the body, allowing the treatment unit 130 to move into the body. Referring to FIG. 6, at the front end portion of the frame 110 a through hole 216 movably guiding and supporting the end portion of the treatment unit 130 and a through hole 217 movably guiding and supporting photographing unit 150 are formed to be long in a length direction. Here, the through holes 216 and 217 may be formed integrally with the frame 210 or at a frame cap 229 mounted at a distal end of frame 210 in the length direction.

Here, the treat unit 130 may be led into the inner portion of the frame 210 through the rear end portion of the frame 210, and then move along the through hole 216 provided at the front end portion or the frame cap 229 to approach the affected part generated in the larynx 103.

Meanwhile, referring to FIG. 6, the medical device for surgery according to another exemplary embodiment of the present invention may includes a frame 210 guiding movement of the treatment unit 130 and preventing the treatment unit 130 from directly contacting inner tissues or preventing the body tissue damage from occurring by the treatment unit 130, wherein the frame 210 may include a fixed frame 228 formed in a straight-line shape and a bent portion 220 connected to the one end of the fixed frame 228 and capable of being deformed or bent. Here, the fixed frame 228 may not be deformed, but the bent portion 220 may be deformed according to a shape of the inner portion of the body or the route of travel to the affected part. That is, the bent portion 220 may be deformed or bent by the force by contact or friction between the bend portion 220 and the inner portion of the body into which the frame 210 is led or inserted. Therefore, the operator may deform the shape of the bent portion 220 or adjust a degree of bending thereof so that the bent portion 220 is fitted with the shape of the inner portion of the body.

As shown in FIG. 6, in the inner portion of the fixed frame 228 of the frame 210 a route of travel for movement of the treatment unit 130 is provided, and the route of travel 211 may have a mounting member 236 connected and fixed to one end of the treatment unit 130. The mounting member 236 contacts the inner surface of the fixed frame 228 to perform sliding movement, and the treatment unit 130 may be transferred by the sliding movement of the mounting member 236.

The fixed frame 228 as shown in FIG. 6 has an open upper portion. However the upper portion of the fixed frame 228 may be shielded by a covering member (not shown) in order to protect the inner portion of the fixed frame 228. Meanwhile, an upper portion 220a and a lower portion 220b of the bent portion 220 may be composed of a plurality of variable members, respectively, so that the bent portion 220 may be bent. That is, although explicitly shown in FIG. 6, the upper portion and lower portion 220b of the bent portion 220 are formed by overlapping many variable members. Therefore, the upper portion and lower portion may seem to have some creases when seen from the outside. In addition, the upper portion 220a and the lower portion 220b of the bent portion 220 may be formed of a plurality of variable members, respectively, so that the bent portion 220 may be bent.

Hereinafter, a structure and operation of the bent portion 220 will be described with reference to drawings.

FIG. 7 is a cross-sectional view partially showing the upper portion 220a of the bent portion of the frame 210 of the medical device for surgery 200 according to another exemplary embodiment of the present invention. Referring to FIG. 7, the bent portion 220 may be formed of a plurality of variable members 221 formed so that they are partially overlapped with each other.

That is, the frame 210 or the bent portion 220 may include a plurality of variable members provided to be overlapped with each so that the shape of the frame 210 or the bent portion 220 may be deformed or bent according to the inner portion of the body contacting the outer surface of the frame while the frame is being led into the inner portion of the body.

As shown in FIG. 7, the variable members 221 may have the same shape and structure. One of both ends of the variable member 221 may be inserted into an inner portion of a neighboring variable and the outer side of the neighboring variable member 221 may be inserted into the other of the variable member 221 member in a length direction of the frame 210 or in a direction of transferring the treatment unit 130. Since neighboring variable members 221 are partially inserted and overlapped with each other, the bent portion 220 may be bent or deformed in the state in which they are in contact with or are connected to each other.

Referring to FIG. 7, the variable member 221 has an approximately inclined shape. However, the present invention is not limited thereto, and any shape that the variable members 221 are able to be bent while maintaining the state of being in contact with and connected to each other and may be applicable. In addition, a variable member's contact portion or contacting surface with the neighboring variable member is formed in a predetermined curved form, such that the variable members may maintain connection to each other in the state of being bent.

As described above, when the bent portion 220 of the frame 210 is formed using a plurality of variable members 221 partially overlapped with each other, the bent portion 220 may be bent or deformed by the force generated by contact between the inner portion of the body and the outer surface of the frame 210 during insertion of the frame 210 into the inner portion of the body. That is, the bent portion 220 may be bent without a separate external force. Therefore, burden that the patient has to keep a specific pose for the surgery may be reduced.

Meanwhile, the variable member 221 may be formed with protrusions 222 protruding toward the route of travel 211. For example, in the case in which the variable member 221 shown in FIG. 7 forms the upper portion 220a of the bent portion 220, the protrusions 222 may protrude toward the route of travel 211 or a bottom surface of the lower portion 220b. That is, the protrusions 222 may be formed to face each other toward the upper portion or lower portion of the route of travel 211.

Here, each protrusion 222 may have a hole 223 at the same position as that of other protrusions, and an operation wire may be inserted in the hole 223. That is, so as to pass through the operation wire 226, each protrusion 222 may be formed with the hole 223 at the same position as that of other protrusions.

In addition, the protrusions may be protruded in a direction perpendicular to the line passing through the center of the route of travel 211. Referring to FIG. 7, the protrusions 222 and the line connecting lower dismal ends of the protrusions 222 to each other, that is, the line parallel to the line passing through the center of the route of travel 211 may be approximately perpendicular to each other. As described above, the protrusions 222 are formed in the direction perpendicular to the line passing through the center of the route of travel 211, whereby the operation wire 226 may be easily inserted into the holes 223. If the protrusions 222 are not formed in the direction perpendicular to the line passing through the center of the route of travel 211, all the holes 223 may not be positioned in the same line, such that it may take a lot of work times to insert the operation wire 226 into the holes 223. It may lead to falling productivity.

Here, the operation wire 226 is the wire that the external force deforming or bending the shape of the bent portion 220 is exerted. So that the bent portion may be deformed or bent, One end of the operation wire 226 may pass through the holes formed in the protrusions 222 and be connected to the frame cap 229 mounted at one end of the frame, and the other may be connected to a bending adjusting portion 260 (see FIG. 8) provided at the other end of the frame 210.

As described above, one end of the operation wire 226 is fixed to the fixed frame cap 229 or the front end portion of the frame 210 and the other is connected to the movable bending adjusting portion 260, thereby making it possible to bend the bent portion 220. That is, when the length of the operation wire 226 passing through the bent portion 220 is reduced by drawing the bending adjusting portion 260, the variable member 221 may be bent or deformed by the reduced length of the operation wire 226.

FIG. 8 is a partially cross-sectional view showing a state in which the bent portion 220 formed of a plurality of variable members are bent. Referring to FIG. 8, one end of each of two operation wires 226a and 226b is connected to the bending adjusting portion 260, wherein the lower operation wire 226b may be in the state of being pulled more than the upper operation wire 226a is. That is, the bent portion may be bent by operating the bending adjusting portion 260 to pull one of the two operation wires more than to pull the other.

Meanwhile, the bending adjusting portion 260 may have a locking means 240 maintaining the deformed state or the bent state of the variable members 221.

The above-mentioned locking means 240 may prevent the frame 210 or the bent portion 220 from unbending by seizing the operation wires 226a and 226b so that the operation wires 226a and 226b do not move in the state in which the frame 210 or the bent portion 220 is bent by the bending adjusting portion 260 pulling one of the operation wires 226a and 226b. That is, the locking means 240 may be the means for fixing or maintaining the lengths of the operation wires 226a and 226b pulled by the bending adjusting portion 260.

Here, the locking means may contact the variable means adjacent to the bending adjusting portion 260 in the state of seizing the operation wire 226. That is, referring to FIG. 8, the locking means 240 may maintain the seizing state of the operation wire 226 while contacting the left-most variable member 227. The locking means 240 may seize the operation wire 226 in the state of contacting the variable member 221 adjacent to the bending adjusting portion 260.

Meanwhile, the locking means may include a clamp (not shown) binding the operation wire 226 or a hook (not shown) caught by a groove (not shown) formed in the operation wire 226. In addition, the locking means 240 may have a sense sensing the length of the operation wire 226. By the sensor, information on how the bent portion 200 is bent may be obtained from the length of the operation wire 226, and the bending adjusting portion 260 and the locking means 240 may be automatically controlled based on the length information described above.

The locking means 240 may not only seize the operation wire 226 but also release the seizure. While the treatment unit 130 is treating the affected part, the locking means 240 may maintain the seizing state of the operation wire 226. When the treatment or surgery is completed or an area to be treated is required to be changed, and so forth, the locking means 240 may operate to release the seizing state.

As described above, since the treatment unit 130 is led into the inner portion of the body using the frame 210 that may be deformed or bent according to the shape of the inner portion of the body, the patient does not have to adopt a specific pose, for example, a pose to lean his or her head backward, or the like. Therefore, applying pressure on a certain part of the body may be prevented, thereby making it possible to prevent incidental body damage from occur.

Although the present invention have been described with reference to specific matters such as specific components, or the like, restricted exemplary embodiments, and drawing, it is only provided to help the general understanding of the present invention. The present invention is not limited thereto, and those skilled in the art will appreciate that various modifications, additions and substitutions are possible from the above description. Therefore, the spirit and scope of the present invention is not limited to the exemplary embodiments described above, and following claims and all other substitutions, alterations and modifications are construed as being included in the spirit of the present invention.

### INDUSTRIAL APPLICABILITY

The present invention may be used for a medical surgery robot, etc.

1. A medical device for surgery comprising:
a frame which is led into a body and the frame's front end portion is fixed adjacent to an affected part, and having a route of travel inside;
at least one treatment unit moving along the route of travel of the frame to be accessible to the affected part and treating the affected part;
a driving unit generating a driving force to drive the at least one treatment unit; and
a control unit controlling the driving unit generated from the control unit to control operation of the treatment unit.

2. The medical device for surgery of claim 1, wherein the frame has a bent portion bending corresponding to the inner portion of the body in a fixed form.

3. The medical device for surgery of claim 1, wherein the frame is formed so as to be deformed or bent according to a shape of the inner portion of the body.

4. The medical device for surgery of claim 3, wherein the frame includes a plurality of variable members provided to be overlapped to each other so that the shape of the frame may be deformed or bent by the shape of inner portion of the body contacting an outer surface of the frame while the frame is being led into the inner portion of the body.

5. The medical device for surgery of claim 4, wherein one of the both ends in a length direction of the frame is inserted into an inside of a neighboring variable member, and an outside of the neighboring variable member is inserted at the other.

6. The medical device for surgery of claim 5, wherein the variable member further has protrusions protruding toward the route of travel, and the protrusion has a hole in which an operation wire is inserted.

7. The medical device for surgery of claim 6, wherein the protrusions are formed to face each other toward the upper and lower portion of the route of travel, or formed to protrude in the direction perpendicular to a line passing through the center of the route of travel.

8. The medical device for surgery of claim 7, wherein one end of the operation wire passes through the holes formed in the protrusions to be connected to a frame cap mounted in the front end of the frame or one end of the frame, and the other is provided to the other end of the frame to be connected to bending adjusting portion adjusting a degree of bending of the variable member.

9. The medical device for surgery of claim 7, wherein the bending adjusting portion has a locking means for maintaining a deformed state or a bent state of the variable member.

10. The medical device for surgery of claim 9, wherein the locking means prevents the frame from unbending by seizing the operation wire in the state in which the frame is bent by the bending adjusting portion pulling one of the operation wires.

11. The medical device for surgery of claim 10, wherein the locking means contacts the variable member adjacent to the bending adjusting portion in the state of seizing the operation wire.

12. The medical device for surgery of claim 10, wherein the locking means includes a clamp seizing the operation wire or a hook caught by a groove formed in the operation wire.

13. The medical device for surgery of any one of claims 9 to 12, wherein the locking wire is able to seize or release the operation wire and maintains the state of seizing the operation wire while the treatment unit is treating the affected part.

14. The medical device for surgery of claim 1, wherein the treatment unit includes:
a treatment portion performing a surgery on the affected part; and
a moving guide portion coupled to the treatment portion by its front and capable of not only having a length direction so as to move along the route of travel but also flexibly operating.

15. The medical device for surgery of claim 14, wherein the moving guide portion is provided as a spring capable of a position change in a length direction and elastic deformation in a lateral direction or made of a flexible plastic or steel material capable of elastic deformation.

16. The medical device for surgery of claim 14, wherein the moving guide portion has a plurality of link members coupling connected in the length direction, and the plurality of link members rotates in multi directions based on a joint portion.

17. The medical device for surgery of claim 14, wherein the treatment portion includes:
a connecting member rotatably coupled to a front end of the moving guide portion in a lateral direction of the moving guide portion and a traverse direction with respect to the moving guide portion;
a rotating member rotatably coupled to the connecting member; and
a treating member coupled to the rotating member and directly performing treatment on the affected part,
the connecting member, the rotating member, and the treating member operate by a driving force generated by the driving unit based on a command signal of the control unit.

18. The medical device for surgery of claim 14, wherein the treating member includes one of a grip for seizing the affected part, scissors for removing the affected part, a knife for cutting out the affected part, a snare for enclosing and cutting out the affected part, a cautery for removing the affected part, and a sucking tube for sucking the removed affected part.

19. The medical device for surgery of claim 18, wherein the driving unit includes:
a front-back moving member moving a mounting member in which a rear end portion of the treatment unit is mounted forward and backward on the route of travel of the frame; and
a wire-type treatment portion driving member connected to the connecting member, the rotating member, and the treating member, portion and controlling operation of a selected member among the connecting member, the rotating member, and the treating member based on the command signal of the control unit.

20. The medical device for surgery of claim 14, further comprising a photographing unit which moves along the route of travel of the frame to approach the affected part and photograph a state of the affected part, and a control unit controls operation of the treatment unit based on photographing information of the photographing unit.
